(19) United States
(12) Patent Application Publication
(10) Pub. No.: US 2006/0078507 A1
(43) Pub. Date: Apr. 13, 2006

(54) Aerosol device containing a composition comprising at least one propellant, at least one acrylic associative polymer, and at least one fixative polymer

(76) Inventors: Charles Gringore, Asnieres sur Seine (FR); Francoise Pataut, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/220,586

(22) Filed: Sep. 8, 2005

Related U.S. Application Data
(60) Provisional application No. 60/620,423, filed on Oct. 21, 2004.

(30) Foreign Application Priority Data
Sep. 9, 2004 (FR) 04 52006

(51) Int. Cl. A61K 8/81

(52) U.S. Cl. 424/47; 424/70.11

(57) ABSTRACT

Disclosed herein is a composition packaged in an aerosol device, containing: at least one propellant; and a hairstyling composition comprising (i) at least one non-polyurethane anionic or non-ionic fixative polymer and (ii) at least one anionic acrylic associative polymer; wherein the at least one propellant is present in an amount greater than 20% by weight relative to the total weight of the composition.

This application claims benefit of U.S. Provisional Application No. 60/620,423, filed Oct. 21, 2004, the contents of which are incorporated herein by reference. This application also claims benefit of priority under 35 U.S.C. § 119 to French Patent Application No. 04 52006, filed Sep. 9, 2004, the contents of which are also incorporated by reference.

The present disclosure relates to an aerosol device containing at least one acrylic associative polymer and at least one fixative polymer, and to a hairstyling method using such a device.

Hairstyling compositions packaged in the form of an aerosol spray generally contain a large fraction of alcohol. Cosmetic products with a high alcohol content, however, are monitored particularly closely, especially in the United States of America, following recent sensitization of public opinion to ecological problems resulting from the emission of volatile organic products into the atmosphere.

A solution which can reduce the quantity of alcohol in formulations or even dispense with it completely is to replace the alcohol with an equivalent quantity of water. However, introducing such large quantities of water into aerosol hair fixing sprays such as lacquers may substantially and undesirably modify the shape of the hair and may deleteriously affect the cosmetic properties. Further, the majority of hydrocarbon type propellants are incompatible with water and their use in compositions with a high water content may therefore be quite difficult, and sometimes impossible.

Hairstyling products are normally used to provide a hairstyle with long-lasting hold. To this end, fixative polymers are introduced into a hydro-alcoholic medium. Packaging into aerosol form allows even distribution over all of the hair. Often the hairstyle is almost immediately fixed and it is difficult if not impossible to sculpt the hair with the hands as the hair is instantaneously fixed.

One of the problems solved by the present disclosure is that of providing a cosmetic hair composition which readily fixes the hair, has good cosmetic properties, for example, a reduction in tackiness, and/or which dries within a reasonable period while distributing itself evenly and rapidly over the hair.

Disclosed herein is a composition packaged in an aerosol device comprising:
a) at least one propellant; and
b) a hairstyling composition comprising at least (i) a non-polyurethane anionic or non-ionic fixative polymer and (ii) at least one anionic acrylic associative polymer.

Also disclosed is a hairstyling method comprising vaporizing the composition contained in the aerosol device over the shaped hair and allowing the treated hair to dry.

Hairstyling Composition

Fixative Polymers

Any anionic or non-ionic fixative polymer or mixture thereof used in the art may be used in compositions in accordance with the present application. The term “fixative polymer” as used herein means any polymer which can endow the hair with hold in a given shape.

The fixative polymers may be soluble in a cosmetically acceptable medium or insoluble in the same medium and, in that case, used in the form of dispersions of solid or liquid particles of polymer (latex or pseudolatex).

Anionic Fixative Polymers

Anionic fixative polymers which are suitable for use in the hairstyling composition described herein include, but are not limited to, polymers comprising at least one group chosen from carboxylic, sulphonic, and phosphoric acid derivative groups and having a number average molecular mass ranging from 500 to 5,000,000. The carboxylic groups may be supplied by unsaturated carboxylic mono- or di-basic acid monomers such as those of formula (I): 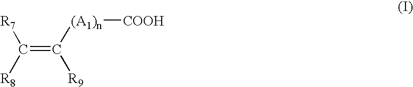
in which:

n is an integer ranging from 0 to 10,

A₁ is a methylene group, optionally bonded to the carbon atom of the unsaturated group or to a neighboring methylene group when n is greater than 1, via a heteroatom such as oxygen or sulphur,

R₇ is chosen from hydrogen, phenyl groups, and benzyl groups,

R₈ is chosen from hydrogen, lower alkyl groups, and carboxyl groups,

R₉ is chosen from hydrogen, lower alkyl groups, —CH₂—COOH groups, phenyl groups, and benzyl groups.

In formula (I) above, a “lower alkyl group” may represent groups comprising from 1 to 4 carbon atoms, for example, methyl and ethyl groups.

Examples of anionic fixative polymers comprising carboxylic groups include, but are not limited to:
A) acrylic acid homopolymers, acrylic acid copolymers, methacrylic acid homopolymers, methacrylic copolymers, and salts thereof, for example, the products sold under the trade names VERSICOL® E and VERSICOL® K by ALLIED COLLOID and ULTRAHOLD® sold by BASF, copolymers of acrylic acid and acrylamide, sold as their sodium salts under the trade names RETEN 421, 423, and 425 by HERCULES, and sodium salts of polyhydroxycarboxylic acids;
B) copolymers of acrylic or methacrylic acid with a monoethylenic monomer such as ethylene, styrene, vinyl esters, acrylic acid esters, and methacrylic acid esters, optionally grafted onto a polyalkylene-glycol such as polyethylene-glycol, and optionally cross-linked. Such polymers are described, for example, in French Patent No. 1 222 944 and German Patent Application No. 2 330 956. Copolymers of this type comprising in their chain an acrylamide motif which may be N-alkylated and/or hydroxyalkylated are described, for example, in Luxembourg Patent Application Nos. 75370 and 75371. Such polymers are also sold under the trade name QUADRAMER by AMERICAN CYANAMID. Other non-limiting examples of suitable copolymers include copolymers of acrylic acid and C₁-C₄ alkyl methacrylate and terpolymers of vinylpyrrolidone, acrylic acid, and C₁-C₂₀ alkyl methacrylate, for example, lauryl, such as those sold by ISP under the trade name ACRYLIDONE® LM, methacrylic acid/ethyl acrylate/tertiobutyl acrylate terpolymers such as the product sold under the trade name LUVIMER® 100 P by BASF, and methacrylic acid/acrylic acid/ethyl acrylate/methyl methacrylate copolymers in aqueous dispersion, sold under the trade name AMERHOLD® DR 25 by AMERCHOL;
C) crotonic acid copolymers, such as those comprising vinyl acetate or propionate motifs in their chain, and other optional monomers such as allyl esters, methallyl esters, and vinyl ethers or vinyl esters of saturated, linear or branched long hydrocarbon chain carboxylic acids such as those comprising at least 5 carbon atoms, said polymers optionally being grafted or cross-linked, or another monomer chosen from vinyl, allyl, and methallyl esters of α- or β-cyclic carboxylic acids. Such polymers are described, for example, in French Patent Nos. 1 222 944, 1 580 545, 2 265 782, 2 265 781, 1 564 110, and 2 439 798. Examples of commercially available products falling into this category include, but are not limited to, Resins 28-29-30, 26-13-14, and 28-13-10 sold by National Starch;
D) copolymers of monounsaturated C₄-C₈ carboxylic acids or anhydrides chosen from:
   copolymers comprising (i) at least one entity chosen from maleic acids, maleic anhydrides, fumaric acids, fumaric anhydrides, itaconic acids, and itaconic anhydrides and (ii) at least one monomer chosen from vinyl esters, vinyl ethers, vinyl halides, phenylvinyl derivatives, and acrylic acid and its esters, the anhydride functions of these copolymers optionally being monoesterified or monoamidified. Such polymers are described, for example, in U.S. Pat. Nos. 2,047,398, 2,723,248, and 2,102,113 and British Patent No. 0 839 805. Examples of commercially available products include, but are not limited to, those sold under the trade names GANTREZ® AN and GANTREZ® ES by ISP; and
   copolymers comprising (i) at least one motif selected from maleic, citraconic, and itaconic anhydride motifs and (ii) at least one monomer chosen from allyl and methallyl esters optionally comprising at least one group chosen from acrylamide, methacrylamide, a-olefin, acrylic, methacrylic ester, acrylic acid, methacrylic acid, and vinylpyrrolidone in their chain, the anhydride functions of said copolymers optionally being monoesterified or monoamidified.

These copolymers are described, for example, in French Patent Nos. 2 350 384 and 2 357 241;
E) polyacrylamides comprising carboxylate groups. Homopolymers and copolymers comprising sulphonic groups may include polymers comprising at least one motif chosen from vinylsulphonic, styrene-sulphonic, naphthalene-sulphonic, and acrylamido-alkylsulphonic motifs. Such polymers may be chosen from, for example:
   salts of polyvinylsulphonic acid having a molecular mass ranging from 1,000 to 100,000 and copolymers with an unsaturated co-monomer, such as acrylic acid, acrylic acid esters, methacrylic acid, methacrylic acid esters, acrylamide and its derivatives, vinyl ethers, and vinylpyrrolidone; and
   polystyrenesulphonic acid salts, such as the sodium salts sold, for example, under the trade names Flexan® 500 and Flexan® 130 by National Starch. Said compounds are described, for example, in French Patent No. 2 198 719 and U.S. Pat. No. 4,128,631. Another non-limiting example of such compounds is polyacrylamidoethylpropanesulphonic acid, sold under the trade name COSMEDIA POLYMER HSP 1180 by HENKEL.

A further example of a suitable anionic fixative polymer is the branched block anionic polymer sold under the trade name Fixate G-100 by NOVEON.

In at least one embodiment, the anionic fixative polymers may be chosen from acrylic acid copolymers such as acrylic acid/ethyl acrylate/N-tertiobutyl acrylamide terpolymers sold under the trade name ULTRAHOLD® STRONG by BASF; copolymers derived from crotonic acid such as vinyl acetate/vinyl tertio-butyl benzoate/crotonic acid terpolymers and crotonic acid/vinyl acetate/vinyl neododecanoate terpolymers sold under the trade name RESYN 28-29-30 by NATIONAL STARCH; copolymers comprising at least one entity chosen from maleic acids, maleic anhydrides, fumaric, acids, fumaric anhydrides, itaconic acids, and itaconic anhydrides and at least one entity chosen from vinyl esters, vinyl ethers, vinyl halides, phenylvinyl derivatives, and acrylic acid and its esters, such as monoesterified methylvinylether/maleic anhydride copolymers sold, for example, under the trade name GANTREZ® by ISP; copolymers of methacrylic acid and methyl methacrylate sold under the trade name EUDRAGIT® L by ROHM PHARMA; copolymers of methacrylic acid and ethyl acrylate sold under the trade name LUVIMER® MAEX or MAE by BASF and vinyl acetate/crotonic acid copolymers sold under the trade name LUVISET CA 66 by BASF; vinyl acetate/crotonic acid copolymers grafted with polyethylene glycol sold under the trade name ARISTOFLEX® by BASF; and the polymer sold under the trade name Fixate G-100 by NOVEON.

In another embodiment of the present disclosure, the anionic fixative polymers of the present disclosure may be chosen from monoesterified methylvinylether/maleic anhydride copolymers sold under the trade name GANTREZ® ES 425 by ISP, acrylic acid/ethyl acrylate/N-tertio-butyl acrylamide terpolymers sold under the trade name ULTRAHOLD® STRONG by BASF, copolymers of methacrylic acid and methyl methacrylate sold under the trade name EUDRAGIT® L by ROHM PHARMA, vinyl acetate/vinyl tertio-butyl benzoate/crotonic acid terpolymers and crotonic acid/vinyl acetate/vinyl neododecanoate terpolymers sold under the trade name Resine 28-29-30 by NATIONAL STARCH, copolymers of methacrylic acid and ethyl acrylate sold under the trade name LUVIMER® MAEX or MAE by BASF, vinylpyrrolidone/acrylic acid/lauryl methacrylate terpolymers sold under the trade name ACRYLIDONE® LM by ISP, and the polymer sold under the trade name Fixate G-100 by NOVEON.

Non-Ionic Fixative Polymers

Examples of non-ionic fixative polymers which may be used in accordance with the present disclosure include, but are not limited to:
polyalkyloxazolines;
vinyl acetate homopolymers;
vinyl acetate copolymers, for example, copolymers of vinyl acetate and acrylic ester, copolymers of vinyl acetate and ethylene, and copolymers of vinyl acetate and a maleic ester, for example dibutyl maleate;
homopolymers and copolymers of acrylic esters, for example, copolymers of alkyl acrylates and alkyl methacrylates, such as the products sold by ROHM & HAAS under the trade name PRIMAL® AC-261 K and EUDRAGIT® NE 30 D, sold by BASF under the trade name 8845, and sold by HOECHST under the trade name APPRETAN® N9212;
copolymers of acrylonitrile and a non-ionic monomer selected, for example, from butadiene and alkyl (meth)acrylates; such as the product sold under the trade name CJ 0601 B by ROHM & HAAS;
styrene homopolymers;
styrene copolymers, for example, copolymers of styrene and alkyl (meth)acrylate, such as the products MOWILITH® LDM 6911, MOWILITH® DM 611, and MOWILITH® LDM 6070 sold by HOECHST, the products RHODOPAS® SD 215 and RHODOPAS® DS 910 sold by RHONE POULENC; copolymers of styrene, alkyl methacrylate, and alkyl acrylate; copolymers of styrene and butadiene; and copolymers of styrene, butadiene, and vinylpyridine;
polyamides;
homopolymers of vinyl lactame other than vinylpyrrolidone homopolymers, such as polyvinylcaprolactame sold under the trade name Luviskol® PLUS by BASF; and
vinyl lactame copolymers, such as a poly(vinylpyrrolidone/vinyl lactame) copolymer sold under the commercial name Luvitec® VPC 55K65W by BASF, poly(vinylpyrrolidone/vinyl acetate) copolymers such as those sold under the trade name PVPVA® S630L by ISP, Luviskol® VA 73, VA 64, VA 55, VA 37, and VA 28 sold by BASF; and poly(vinylpyrrolidone/vinyl acetate/vinyl propionate) terpolymers such as, for example, the product sold under the trade name Luviskol® VAP 343 by BASF.

In at least one embodiment of the present disclosure, the alkyl groups of the non-ionic polymers disclosed herein may contain from 1 to 6 carbon atoms.

In accordance with the present disclosure, it is also possible to use anionic or non-ionic graft silicone type fixative polymers comprising a polysiloxane portion and a portion comprising an organic non-silicone chain, one of the two portions constituting the principal chain of the polymer and the other being grafted onto said principal chain. Such polymers are described, for example, in European Patent Application Nos. 0 412 704 A, 0 412 707 A, 0 640 105 A, and 0 582 152 A, International Patent Application Publication Nos. WO 95/00578 and WO 93/23009, and U.S. Pat. Nos. 4,693,935, 4,728,571, and 4,972,037.

Such polymers may be copolymers obtained, for example, by radical polymerization of a mixture of monomers comprising:
a) 50% to 90% by weight of tertiobutyl acrylate;
b) 0 to 40% by weight of acrylic acid;
c) 5% to 40% by weight of silicone macromers of formula: 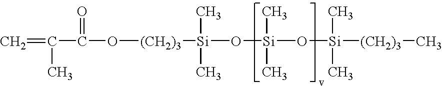
   in which v is a number ranging from 5 to 700, the percentages by weight being calculated with respect to the total weight of monomers.

Other examples of graft silicone polymers include, but are not limited to, polydimethylsiloxanes (PDMS) onto which are grafted, via a thiopropylene type connecting link, mixed polymer motifs of the poly((meth)acrylic acid) and poly(alkyl (meth)acrylate) type, and polydimethylsiloxanes (PDMS) onto which are grafted, via a thiopropylene type connecting link, polymer motifs of the isobutyl poly(meth)acrylate type.

Another silicone fixative polymer suitable for use in the presently disclosed compositions is the product Luviflex® Silk sold by BASF.

The at least one fixative polymer may be present in the composition in an amount ranging from 0.1% to 20% by weight, for example, from 0.5% to 10% by weight with respect to the total weight of the composition contained in the aerosol device (propellant+hairstyling composition).

Anionic Acrylic Associative Polymers

As used herein, the term “associative polymer” means any amphiphilic polymer comprising at least one fatty chain, i.e., a hydrophobic portion and at least one hydrophilic portion.

The molecules of said polymers may be capable of combining with each other and/or with molecules of associative agents such as surfactants, resulting in particular properties.

The number of hydrophobic portions may be smaller compared with the remainder of the polymeric chain, and the hydrophobic portions may be located laterally of the chain and be distributed in a random manner (random copolymers) or be distributed in the form of blocks or grafts (block copolymers or sequence copolymers).

Water-soluble polymers or hydrodispersible polymers may also be used. In at least one embodiment, the amphiphilic polymers used in accordance with the present disclosure are not cross-linked.

The anionic associative polymers of the present disclosure may comprise, as monomers, at least one unsaturated carboxylic acid. Unsaturated carboxylic acids which may be used include, but are not limited to, acrylic, methacrylic, crotonic, itaconic, and maleic acids.

As used herein, the expression “fatty chain” means a linear or branched hydrocarbon group comprising from 8 to 30 carbon atoms.

Non-limiting examples of anionic acrylic associative polymers include:
copolymers comprising at least one hydrophilic motif and at least one fatty chain allyl ether motif, the hydrophilic motif of which corresponds to a vinyl carboxylic acid, for example, acrylic acid, methacrylic acid, and mixtures thereof, and the fatty chain allyl ether motif of which corresponds to a monomer of formula (V):
   CH₂═CR′CH₂OBₙR (V)
   in which:
R′ is chosen from hydrogen and CH₃;
B is an ethyleneoxy radical;
n is an integer ranging from 0 to 100;
R is a hydrocarbon radical selected from alkyl, arylalkyl, aryl, alkylaryl, and cycloalkyl radicals comprising from 8 to 30 carbon atoms, for example, from 10 to 24 carbon atoms, or from 12 to 18 carbon atoms.

In at least one embodiment, the motif of formula (V) is a motif in which R′ is hydrogen, n is equal to 10, and R is a stearyl radical (C₁₈).

Anionic associative polymers of this type and their preparation by an emulsion polymerization method have been described, for example, in European Patent No. 0 216 479.

In one embodiment of the present disclosure, the anionic associative polymers are chosen from polymers comprising from 20% to 60% by weight of acrylic acid and/or methacrylic acid, from 5% to 60% by weight of lower alkyl (meth)acrylates, from 2% to 50% by weight of a fatty chain allyl ether of formula (V), and from 0 to 1% by weight of a cross-linking agent chosen from well known copolymerizable unsaturated polyethylenic monomers, such as diallyl phthalate, allyl (meth)acrylate, divinylbenzene, (poly)ethyleneglycol dimethacrylate, and methylene-bis-acrylamide.

Examples of such polymers include the cross-linked terpolymers sold by ALLIED COLLOIDS under trade names SALCARE SC 80® and SALCARE SC90®, which are 30% aqueous emulsions of a cross-linked terpolymer of methacryic acid, ethyl acrylate, and steareth-10-allyl ether (40/50/10), and the products RHOEVIS-CR, -CR3 and -CRX sold by ALLIED COLLOIDS;
methacrylic acid/alkyl acrylate/polyoxyethyenated lauryl acrylate terpolymers, such as RHEO 2000 sold by COATEX;
methacrylic acid/alkyl acrylate/polyoxyethylenated stearyl methacrylate copolymers, such as ACRYSOL 22 (or ACULYN 22 or ACRYSOL ICS), ACRYSOL 25, and DW-1206A sold by ROHM & HAAS;
methacrylic acid/alkyl acrylate/polyoxyethylenated nonylphenol acrylate copolymers such as RHEO 3000 sold by COATEX;
methacrylic acid/butyl acrylate/hydrophobic monomer copolymers comprising a fatty chain such as the 8069-146A product sold by NATIONAL STARCH; and
copolymers comprising at least one unsaturated olefinic carboxylic acid type hydrophilic motif and at least one C₁₀-C₃₀ alkyl ester of an unsaturated carboxylic acid type hydrophobic motif.

In at least one embodiment, such polymers may be chosen from those comprising an unsaturated olefinic carboxylic acid type hydrophilic motif corresponding to a monomer of the following formula: 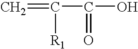
in which R₁ is chosen from hydrogen, CH₃, and C₂H₅, i.e., acrylic, methacrylic, or ethacrylic acid motifs, and unsaturated carboxylic acid alkyl (C₁₀-C₃₀) ester type hydrophobic motif, corresponding to a monomer of the following formula: 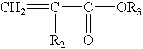
in which:
R₂ is chosen from hydrogen, CH₃, and C₂H₅, i.e. acrylate, methacrylate, or ethacrylate motifs; and
R₃ is chosen from C₁₀-C₃₀ alkyl radicals, for example, C₁₂-C₂₂ radicals. In one embodiment, R₂ may be chosen from hydrogen, to form an acrylate motif, and CH₃, to form a methacrylate motif.

Examples of (C₁₀-C₃₀) alkyl esters of unsaturated carboxylic acids for use in the present disclosure include, but are not limited to, lauryl acrylate, stearyl acrylate, decyl acrylate, isodecyl acrylate, dodecyl acrylate, lauryl methacrylate, stearyl methacrylate, decyl methacrylate, isodecyl methacrylate, and dodecyl methacrylate.

Examples of anionic polymers of this type and the preparation thereof are described, for example, in U.S. Pat. Nos. 3,915,921 and 4,509,949.

Other non-limiting examples of this type of anionic associative polymer include polymers formed from a mixture of monomers comprising:
(i) acrylic acid;
(ii) esters of formula (VII) as described above, in which R₂ is chosen from hydrogen and CH₃, R₃ is chosen from alkyl radicals comprising from 12 to 22 carbon atoms; and
(iii) at least one cross-linking agent, which may be chosen from well known copolymerizable polyethylenic unsaturated monomers, such as diallyl phthalate, allyl (meth)acrylate, divinylbenzene, (poly)ethyleneglycol dimethacrylate, and methylene-bis-acrylamide.

Further examples of anionic associative polymers which may be used in accordance with the present disclosure include those comprising from 95% to 60% by weight of acrylic acid (hydrophilic motif), from 4% to 40% by weight of C₁₀-C₃₀ alkyl acrylate (hydrophobic motif), and from 0 to 6% by weight of cross-linking polymerizable monomer, and those comprising from 98% to 96% by weight of acrylic acid (hydrophilic motif), from 1% to 4% by weight of C₁₀-C₃₀ alkyl acrylate (hydrophobic motif), and from 0.1% to 0.6% by weight of cross-linking polymerizable monomer such as those described above.

In one embodiment, the anionic associative polymers may be chosen from the products sold by GOODRICH under the trade names PEMULEN TR1®, PEMULEN TR2®, and CARBOPOL 1382®, and the product sold by S.E.P.P.I.C. under the trade name COATEX SX®.

Additional non-limiting examples of suitable anionic associative polymers include:
(meth)acrylic acid/ethyl acrylate/C₈-C₂₂ alkyl acrylate copolymers, such as ACUSOL 823 sold by ROHM & HAAS and IMPERON R sold by HOECHST;
terpolymers of acrylic acid/C₈-C₃₀ alkyl acrylate, for example, C₁₅/polyethylene glycol (for example, 20 to 30 moles of ethylene oxide) such as DAPRAL GE 202 sold by AKZO;
maleic anhydride/C₃₀-C₃₈ α-olefin/alkyl maleate terpolymers, such as the (copolymer of maleic anhydride/C₃₀-C₃₈ α-olefin/isopropyl maleate) product sold under the trade name PERFORMA V 1608® by NEWPHASE TECHNOLOGIES; and
acrylic copolymers with at least one polyurethane motif, such as acrylic terpolymers comprising:
   (a) 20% to 70% by weight of a carboxylic acid containing an α,β-monoethylenic unsaturated bond;
   (b) 20% to 80% by weight of a monomer with an α,β-monoethylenic unsaturated bond which is not a surfactant and is different from (a); and
   (c) 0.5% to 60% by weight of a non-ionic mono-urethane which is the reaction product of a monohydric surfactant with a mono-isocyanate having a monoethylenic unsaturated bond;
   such as those described in European Patent Application No. 0 173 109, for example, the polymer described in Example 3, namely a methacrylic acid/methyl acrylate/dimethyl meta-isopropenyl benzyl isocyanate of ethoxylated (40 OE) behenyl alcohol in 25% aqueous dispersion. An analogous product which may be cited is the product ADDITOL VXW 1312 sold by HOECHST.

In one embodiment of the present disclosure, the associative polymers may be chosen from:
acrylic acid/ethyl C₁-C₈ acrylate/stearyl methacrylate, polyoxyethylenated, for example, using 20 moles of ethylene oxide, such as the products sold under the trade names “ACRYSOL ICS, ACRYSOL ZZ, and ACULYN 22” by ROHM & HAAS.

Depending on their nature, the associative polymers disclosed herein may be used in the form of aqueous solutions or in the form of aqueous dispersions.

Aerosol

The propellant may be any liquefiable gas which is normally used in aerosol devices, such as dimethylether, C₃-C₅ alkanes, 1,1-difluoroethane, mixtures of dimethyl ether and C₃-C₅ alkanes, and mixtures of 1,1-difluoroethane and dimethylether and/or C₃-C₅ alkanes. In at least one embodiment, the propellant is dimethylether. The propellant may be present in an amount greater than 20% by weight, and may range, for example, from 20% to 70% by weight, or from 20% to 50% by weight of the total weight of the mixture (propellant (a)+hairstyling composition (b)) contained in the aerosol device.

Although the hairstyling compositions contained in the aerosol device of the present disclosure are, in at least one embodiment, free of lower alcohols, they may contain, for example, up to 50%, or up to 25% by weight, with respect to the total weight of the composition contained in the aerosol device, of one or more lower alcohols such as ethanol or isopropanol.

The hairstyling composition of the present disclosure may also contain at least one adjuvant selected from silicones in a soluble, dispersed or micro-dispersed form; non-ionic surfactants; anionic surfactants; cationic surfactants; amphoteric surfactants; additional polymers other than the fixative polymers and the associative polymers used in the compositions disclosed herein; ceramides; pseudo-ceramides; vitamins; pro-vitamins such as panthenol; vegetable oils; animal oils; mineral oils; synthetic oils; waxes other than ceramides and pseudo-ceramides; hydrosoluble or liposouble, silicone or non silicone sunscreens; solid particles such as mineral and organic pigments, which may or may not be colored; pearlizing agents, opacifying agents; glitter; active particles; coloring agents; sequestrating agents; plasticizers; solublizing agents; acidifying agents; alkalinization agents; neutralizing agents; mineral and organic thickening agents; anti-oxidants; hydroxyacids; penetrating agents; fragrances; and preservatives.

These additives may be present in the compositions disclosed herein in an amount ranging from 0 to 20% by weight with respect to the total weight of the composition contained in the aerosol device.

In the hairstyling compositions contained in the aerosol device, the water content may range from 20% to 80% by weight with respect to the total weight of the composition contained in the aerosol device.

Clearly, the skilled person will take care to select any complementary additives and/or their quantities such that the intrinsic advantageous properties of the present disclosure are not changed by the envisaged adjuvants.

When used under the conditions described above, i.e., in an aerosol device, the associative polymers of the present disclosure may facilitate distribution of the hairstyling solution over the hair and reduce the tacky effect of the fixative polymers disclosed herein.

This particular association means that the hairstyle can be shaped by working with the fingers without the instantaneous hardening and fixing conventionally associated with fixing products. It may be applied to both wet and dry hair.

In the context of application to wet hair, it may be allowed to air dry or may be blow-dried. The resulting hairstyle may be flexible and natural.

Introducing specific additives of the polyol type into this type of formulation may, for example, produce a wet-look effect, i.e., may give the hairstyle a long-lasting “fresh from the shower” appearance. The resulting hairstyle may be flexible and natural.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below.

### EXAMPLE

The following hydroalcoholic aerosol spray was prepared:

| Ingredient | g/100 g active material |
|---|---|
| PVP | 2.27 |
| Aculyn 22 | 0.65 |
| 2-amino-2-methyl-1-propanol | 0.6 |
| Water | 36.40 |
| Glycerol | 10.40 |
| Ethanol | 15.22 |
| DME | 35 |

When sprayed onto natural wet hair, the above composition was easily distributed over the hair without being tacky on the fingers and after drying, gave the hair a long-lasting wet-look, with flexible, natural hold.

What is claimed is:

1. A composition packaged in an aerosol device, comprising:
(a) at least one propellant; and
(b) a hairstyling composition comprising at least (i) a non-polyurethane anionic or non-ionic fixative polymer and (ii) at least one anionic acrylic associative polymer
wherein the at least one propellant is present in an amount greater than 20% by weight relative to the total weight of the composition.

2. The composition of claim 1, wherein the at least one anionic acrylic associative polymer is selected from:
polymers comprising at least one hydrophilic motif and at least one fatty chain allyl ether motif, the hydrophilic motif of which corresponds to a vinyl carboxylic acid monomer, and the fatty chain allyl ether motif of which corresponds to a monomer of formula (V):
CH₂═CR′CH₂OBₙR (V)
in which:
R′ is chosen from hydrogen and CH₃;
B is an ethyleneoxy radical;
n an integer ranging from 0 to 100;
R is a hydrocarbon radical selected from alkyl, arylalkyl, aryl, alkylaryl, and cycloalkyl radicals comprising from 8 to 30 carbon atoms;
methacrylic acid/alkyl acrylate/polyoxyethyenated lauryl acrylate terpolymers;
methacrylic acid/C₁-C₈ alkyl acrylate/polyoxyethylenated stearyl methacrylate copolymers;
methacrylic acid/ethyl acrylate/polyoxyethylenated nonylphenol acrylate copolymers;
acrylic acid/polyoxyethylenated stearyl mono-itaconate copolymers or acrylic acid/polyoxyethylenated cetyl mono-itaconate copolymers;
copolymers comprising at least one unsaturated olefinic carboxylic acid type hydrophilic motif and at least one C₁₀-C₃₀ alkyl ester of an unsaturated carboxylic acid type hydrophobic motif;
maleic anhydride/C₃₀-C₃₈ α-olefin/alkyl maleate terpolymers; and
acrylic copolymers with at least one polyurethane motif.

3. The composition of claim 2, wherein when the at least one anionic acrylic associative polymer is chosen from polymers comprising at least one hydrophilic motif and at least one fatty chain allyl ether motif corresponding to the monomer of formula (V), the at least one hydrophilic motif corresponds to an acrylic acid or methacryic acid monomer or mixtures thereof.

4. The composition of claim 2, wherein, in formula (V), R is a hydrocarbon radical selected from alkyl, arylalkyl, aryl, alkylaryl, and cycloalkyl radicals comprising from 10 to 24 carbon atoms.

5. The composition of claim 2, wherein, in formula (V), R is a hydrocarbon radical selected from alkyl, arylalkyl, aryl, alkylaryl, and cycloalkyl radicals comprising from 12 to 18 carbon atoms.

6. The composition of claim 1, wherein the at least one anionic acrylic associative polymer is selected from acrylic acid/ethyl acrylate/polyoxyethylenated stearyl methacrylate terpolymers.

7. The composition of claim 1, wherein the at least one anionic acrylic associative polymer is present in an amount ranging from 0.01% to 30% by weight relative to the total weight of the composition contained in the aerosol device.

8. The composition of claim 7, wherein the at least one anionic acrylic associative polymer is present in an amount ranging from 0.1% to 20% by weight relative to the total weight of the composition contained in the aerosol device.

9. The composition of claim 8, wherein the at least one anionic acylic associative polymer is present in an amount ranging from 0.5% to 15% by weight relative to the total weight of the composition contained in the aerosol device.

10. The composition of claim 1, wherein the at least one anionic fixative polymer is chosen from homopolymers and copolymers of acrylic and methacrylic acids and their salts, copolymers of crotonic acid, copolymers of monounsaturated C₄-C₈ carboxylic acids and anhydrides, polyacrylamides containing carboxylate groups, homopolymers or copolymers containing sulphonic groups, and anionic graft silicone polymers.

11. The composition of claim 1, wherein the at least one non-ionic fixative polymer is chosen from polyalkyloxazolines, homopolymers and copolymers of vinyl acetate, homopolymers and copolymers of acrylic esters, copolymers of acrylonitrile, homopolymers and copolymers of styrene, homopolymers of vinyl lactame other than homopolymers of vinylpyrrolidone, copolymers of vinyl lactame and non-ionic graft silicone polymers.

12. The composition of claim 1, wherein the at least one fixative polymer is present in an amount ranging from 0.1% to 20% by weight relative to the total weight of the composition contained in the aerosol device.

13. The composition of claim 12, wherein the at least one fixative polymer is present in an amount ranging from 0.5% to 10% by weight relative to the total weight of the composition contained in the aerosol device.

14. The composition of claim 1, further comprising 20% to 80% by weight of water relative to the total weight of the composition contained in the aerosol device.

15. The composition of claim 1, wherein the at least one propellant is present in an amount ranging from 20% to 70% by weight relative to the total weight of the composition contained in the aerosol device.

16. The composition of claim 15, wherein the at least one propellant is present in an amount ranging from 20% to 50% by weight relative to the total weight of the composition contained in the aerosol device.

17. The composition of claim 1, wherein the at least one propellant is chosen from dimethylether, difluoroethane, C₃-C₅ alkanes, and mixtures thereof.

18. The composition of claim 17, wherein the at least one propellant is dimethyl ether.

19. The composition of claim 1, further comprising at least one adjuvant chosen from silicones in a soluble, dispersed, micro-dispersed form; non-ionic surfactants; anionic surfactants; cationic surfactants; amphoteric surfactants; additional polymers other than the fixing or associative polymers recited in claim 1; ceramides; pseudo-ceramides; vitamins; pro-vitamins; vegetable oils; animal oils; mineral oils; synthetic oils; waxes other than ceramides and pseudo-ceramides; hydrosoluble or liposoluble, silicone or non silicone sunscreens; solid particles; mineral and organic pigments which may or may not be colored; pearlizing agents; opacifying agents; glitter; active particles; coloring agents; sequestrating agents; plasticizers; solubilizing agents; acidifying agents; alkalinizing agents; neutralizing agents; mineral and organic thickening agents; anti-oxidants; hydroxy acids; penetrating agents; fragrances; and preservatives.

20. A hairstyling method comprising
applying to shaped hair a composition packaged in an aerosol device and comprising:
(a) at least one propellant; and
(b) a hairstyling composition comprising at least (i) a non-polyurethane anionic or
non-ionic fixative polymer and (ii) at least one anionic acrylic associative polymer wherein the at least one propellant is present in an amount greater than 20% by weight relative to the total weight of the composition, and
allowing the hair to dry.
